(19) United States
(12) Patent Application Publication
(10) Pub. No.: US 2011/0029061 A1
(43) Pub. Date: Feb. 3, 2011

(54) CARINA MODIFICATION CATHETER

(75) Inventors: Yong Chul Ahn, Gyeonggi-do (KR); Yangsoo Jang, Seoul (KR); Sung Il Baik, Gyeonggi-do (KR)

Correspondence Address: SWANSON & BRATSCHUN, L.L.C., 8210 SOUTHPARK TERRACE, LITTLETON, CO, 80120, US

(73) Assignee: INDUSTRY-ACADEMIC COOPERATION FOUNDATION, YOUNSEI UNIVERSITY

(21) Appl. No.: 12/934,961

(22) PCT Filed: Mar. 26, 2009
(86) PCT No.: PCT/KR09/01535

§ 371 Date: Sep. 27, 2010

(30) Foreign Application Priority Data
Mar. 27, 2008 (KR) 10-2008-0028310

(51) Int. Cl. A61F 2/82

(52) U.S. Cl. 623/1.11

(57) ABSTRACT

The present invention relates to a carina modification catheter capable of removing a protrusion (carina) of a stent installed in a branched blood vessel, and more particularly, to a catheter which can remove a protrusion of a stent in a branched blood vessel by being easily inserted into a main blood vessel and the branched blood vessel when adjustment of the position of stents installed in the main blood vessel and the branched blood vessel is required, thereby completely covering a branched-portion de novo lesion and reducing the possibility of instent-restenosis (ISR) in the blood vessel after the operation.

### TECHNICAL FIELD

The present invention generally relates to a carina modification catheter capable of removing a protrusion of a stent installed in a branched blood vessel, and more particularly, to a catheter which can remove a protrusion of a stent in a branched blood vessel by being easily inserted into a main blood vessel and the branched blood vessel.

### BACKGROUND ART

For Percutaneous Coronary Intervention (PCI), which is a medical treatment for enlarging a vessel whose diameter is narrowed due to stenosis or occlusion, stents and balloon catheters have been widely used.

As shown in FIG. 1, when stenosis occurs in a boundary portion between a main blood vessel 10 and a branched blood vessel 11 branching out from the main blood vessel 10, stents 210 and 220 are installed in the main blood vessel 10 and the branched blood vessel 11, respectively.

In many of the patients into which the stents 210 and 220 are inserted, the stent 220 is not correctly positioned in a branched portion or some region of the stent 220 inserted into the branched blood vessel 11 is positioned in the stent 210 inserted into the main blood vessel 10.

In this state, a protrusion (a portion positioned in the stent 210 inserted into the main blood vessel 10) of the stent 220 inserted into the branched blood vessel 11 is called a carina (see a portion A of FIG. 1).

That is, if a new de novo lesion occurs in the bifurcation, it is difficult to expect effective treatment in the lesion portion merely from a surgical operation with current stents being in the shape of a metal mesh coated with medicine, and even after the surgical operation, instent-restenosis (ISR) may occur in the branched portion, requiring retreatment.

### DISCLOSURE OF INVENTION

### Technical Problem

The present invention is designed to solve the foregoing problems and an object of the present invention is to provide a carina modification catheter which can quickly adjust the position of stents inserted into a main blood vessel and a branched blood vessel at the same time while removing a protrusion of the stent inserted into the branched blood vessel when readjustment of the position of those stents is required.

Another object of the present invention is to provide a carina modification catheter having high durability due to the structure thereof, in which a support member is inserted into a central axis of the catheter.

### TECHNICAL SOLUTION

To achieve the foregoing objects, there is provided a carina modification catheter comprising a main body and a first guide portion and a second guide portion which are fixed to the main body, respectively.

Herein, each of the first guide portion and the second guide portion preferably comprises a fixed portion fixed to the main body and a branched portion extending from the fixed portion and bent at a front end portion of the main body, the front end portion being capable of pressurizing a protrusion portion of a stent inserted into a branched blood vessel.

### ADVANTAGEOUS EFFECTS

As described above, the carina modification catheter according to the present invention pressurizes a stent protrusion portion formed in case of stenting in a branched blood vessel de novo lesion (Provisional T-stent, Crush stent, V-stent, Y-stent, etc.) by means of the front end portion of the main body of the catheter to urge the protrusion portion close to the blood vessel, thereby improving the blood flow.

Furthermore, the carina modification catheter according to the present invention is provided with guide portions at both its sides to guide the catheter, in which two guide wires, when being inserted, do not interfere with each other through blocking plates of the main body, thereby facilitating insertion of the guide wires and enabling a stable operation.

### BRIEF DESCRIPTION OF DRAWINGS

FIG. 1 illustrates a carina (a protrusion) formed when a stent is placed in a branched blood vessel according to the prior art;

FIG. 2 is a cross-sectional view of a carina modification catheter according to an embodiment of the present invention;

FIG. 3 is a cross-sectional view of a carina modification catheter according to an embodiment of the present invention, showing a state where guide wires are inserted through guide portions of the catheter;

FIGS. 4 to 6 are views for explaining a method of pressurizing a carina formed in a branched blood vessel by means of the catheter according to the present invention;

FIG. 7 shows a stent placed in a branched blood vessel, which has an improved structure from which a protrusion is removed by pressurizing the carina portion by means of the carina modification catheter according to the present invention.

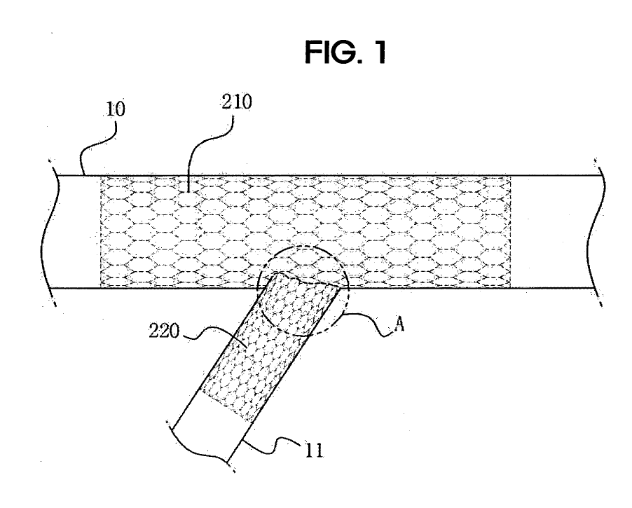

### BEST MODE FOR CARRYING OUT THE INVENTION

Hereinafter, the present invention will be described in more detail with reference to the accompanying drawings. The accompanying drawings show an exemplary embodiment of the present invention and are provided to describe the present invention in more detail, without limiting the technical scope of the present invention.

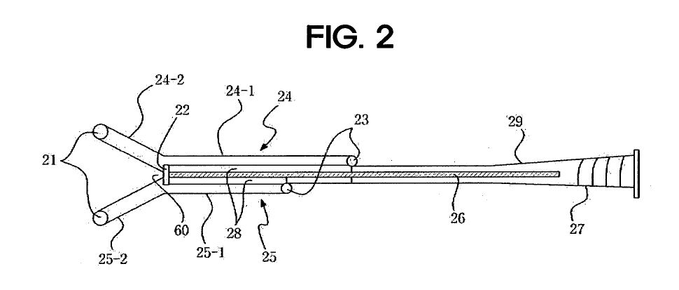

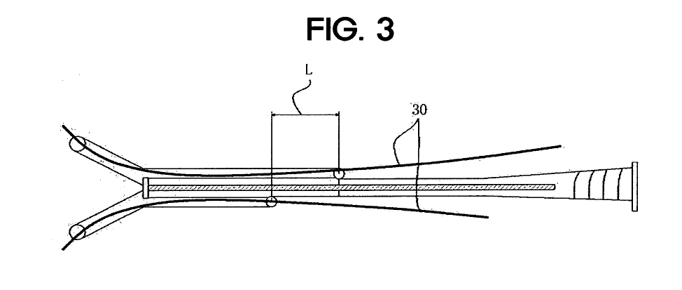

FIG. 2 is a cross-sectional view of a carina modification catheter according to an embodiment of the present invention, and FIG. 3 is a cross-sectional view of a carina modification catheter (hereinafter, referred to as a catheter for short) according to an embodiment of the present invention, showing a state where guide wires are inserted along guide portions of the catheter.

A carina modification catheter according to an embodiment of the present invention comprises a main body 29 and first and second guide portions 24 and 25, which are fixed onto an outer circumferential surface of the main body 29, respectively.

Herein, the first and second guide portions 24 and 25 comprise fixed portions 24-1 and 25-1 fixed on the outer circumferential surface of the main body 29 and branched portions 24-2 and 25-2 extending outward from a front end portion 60 of the main body 29, respectively.

The two branched portions 24-2 and 25-2 are preferably inclined at an acute angle with respect to the front end portion 60 of the main body 29. The fixed portions 24-1 and 25-1 of the first and second guide portions 24 and 25 may be bonded to the outer circumferential surface of the main body 29, and may be tubes formed of polyamide, the tubes having stainless meshes inserted thereinto.

The fixed portions 24-1 and 25-1 have to be able to maintain predetermined rigidity, and are formed of hard materials. A reinforcing material such as stainless meshes may be inserted thereinto.

The branched portions 24-2 and 25-2 may be tubes formed of polyamide and are preferably formed of soft materials. The two tubes have different properties (the fixed portion and the branched portion) and may be bonded with heat applied thereto by using a welder.

The fixed portions 24-1 and 25-1 are preferably made of hard materials to provide a predetermined supporting force during insertion of the catheter into the blood vessel, and the main body 29 preferably comprises a support member 26 therein along its longitudinal direction to secure a high supporting force in the longitudinal direction of the main body 29 during insertion of the catheter.

The support member 26 is named a core wire and may be made of nitinol composed of 43.9% titanium (Ti) and 56.1% nickel (Ni). For the support member 26, a guide wire of 0.014 inch (0.35 mm) is preferably used.

As shown in FIGS. 2 and 3, the lengths of the fixed portions 24-1 and 25-1 of the first and second guide portions 24 and 25 may be different from each other (see a portion L of FIG. 3). When a surgical operation is performed through the fixed portions 24-1 and 25-1 having different lengths, a wire positioned in the main blood vessel and a wire positioned in the branched blood vessel can be easily distinguished from each other. For example, a difference between the lengths of the fixed portions 24-1 and 25-1 of the first and second guide portions 24 and 25 may be, without being limited to, in a range from 7 cm to 12 cm, and preferably in a range from 9 cm to 11 cm, and more preferably about 10 cm.

With such a difference in length, the catheter can secure a high supporting force up to the ends of the branched portions 24-2 and 25-2 with respect to the guide wires 30 which may be positioned inside the first and second guide portions 24 and 25.

The branched portions 24-2 and 25-2 of the first and second guide portions 24 and 25 are preferably formed of soft materials to enable flexible insertion according to a coupling angle between the main blood vessel and the branch blood vessel, and are preferably formed in a Y tube-like shape.

The branched portion 24-2 of the first guide portion 24 and the branched portion 25-2 of the second guide portion 25, which form an acute angle therebetween at the front end portion 60 of the main body 29, preferably have a length of 2 mm to 5 mm. When each of the branched portions 24-2 and 25-2 has a length of less than 2 mm, it has difficulty in functioning as the branched portion. When the length exceeds 5 mm, damage to the blood vessel may be caused due to excessive length of the branched portion.

The first and second guide portions 24 and 25 comprising the fixed portions 24-1 and 25-1 and the branched portions 24-2 and 25-2, respectively, are hollow tube-like members having both ends 21 and 23 open, and the guide wires 30 can pass through the inside of the first and second guide portions 24 and 25. The open ends 21 and 23 comprise the ends 21 of the branched portions 24-2 and 25-2 into which the guide wires 30 are initially inserted and the ends 23 of the fixed portions 24-1 and 25-1 through which the guide wires 30 passing through the guide portions 24 and 25 go out.

Each of the first and second guide portions 24 and 25 preferably has a diameter larger than the diameter of each of the guide wires 30 such that the guide wires 30 can be inserted into and then pass through the first and second guide portions 24 and 25. For example, when the guide wires 30 having a diameter of 0.014 inches are used, the diameter of each of the first and second guide portions 24 and 25 may be 0.015 inches to 0.016 inches.

A marking unit 22 may be disposed in the front end portion 60 of the main body 29, and the marking unit 22 may be a radio marker made of metal or resin which facilitates recognition of the position of the catheter inserted into the blood vessel. The recognition of the position of the catheter is very important in pressurizing a stent protrusion in the branched blood vessel.

The radio marker is also called a marker band and, for example, may be made of a combination of 90% platinum and 10% iridium.

In order to prevent the main body 29 of the catheter from being bent when the stent protrusion in the branched blood vessel is pressurized, the radio marker, which is the marking unit 22, is positioned at an end of the support member 26 of the main body 29 and is bonded to a boundary point between the fixed portions 24-1 and 25-1 and the branched portions 24-2 and 25-2 of the first and second guide portions 24 and 25. The marking unit 22 may also be used as a pressurizing means and an operator can accurately recognize the position of the catheter through the radio marker when the part of a patient undergoing an operation is illuminated with fluoroscopy.

The main body 29 may comprise a housing made of a soft material, the support member 26 disposed in the longitudinal direction in the housing, and first and second blocking plates 28 disposed between the support member 26 and the housing and corresponding to the fixed portions 24-1 and 25-1 of the first and second guide portions 24 and 25, respectively.

The fixed portions 24-1 and 25-1 of the first and second guide portions 24 and 25 are bonded to the main body 29 structured as described above, and the main body 29 serves as a support and a grip and its portion serving as a grip is also called a hub 27 which may be made of polycarbonate.

The soft material of the housing may be polyamide. The support member 26 has to maintain predetermined rigidity during a process of insertion into or withdrawal from the blood vessel during an operation with the catheter, and thus is preferably made of a hard material. For example, the support member 26 may be a wire having a predetermined diameter.

The first and second blocking plates 28, which correspond to the fixed portions 24-1 and 25-1 of the first and second guide portions 24 and 25, respectively, may be positioned between the support member 26 and the housing. Thus, although the guide wires 30 are bent by a pressurizing force, they can be inserted without interfering with each other, such as blocking each other, by the first and second blocking plates 28, and can pressurize a protrusion formed in a stent inserted into the branched blood vessel with a given proper supporting force.

To improve treatment efficacy on a bifurcation de novo lesion which has not yet been solved in current coronary artery intervention, the following operation may be performed.

After respective stents are inserted into the bifurcation de novo lesion by using a stent catheter, 2 guide wires of 0.014 inch diameter are in-situ positioned in the main blood vessel and the branched blood vessel. The in-situ positioned guide wires then perform a function of guiding the carina modification catheter to a desired operation part.

During the operation, by using the catheter according to the present invention, the two guide wires are inserted into the ends 21 of the branched portions 24-2 and 25-2 of the first and second guide portions 24 and 25 and go out through the ends 23 of the fixed portions 24-1 and 25-2 of the first and second guide portions 24 and 25. Although the respective guide wires 30 are bent by the pressurizing force, they can be inserted without interfering with each other, such as blocking each other, by the first and second blocking plates 28 and pressurize the protrusion formed in the stent inserted into the branched blood vessel with a given proper supporting force.

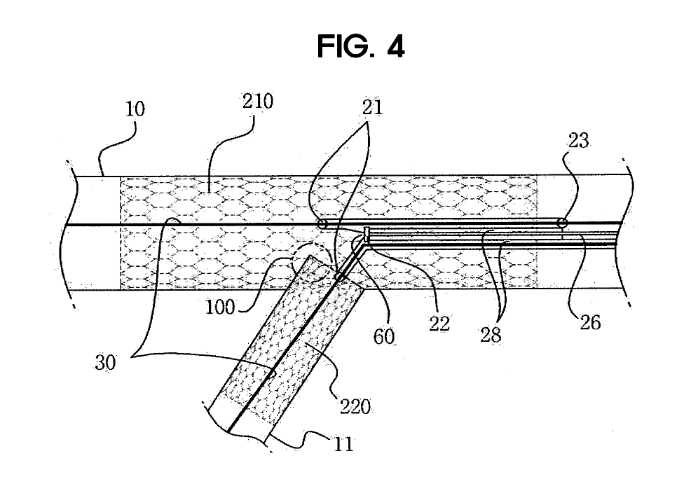

FIGS. 4 to 6 are views for explaining a method of pressurizing a carina formed in a branched blood vessel by means of the catheter according to the present invention.

Referring to FIG. 4, when positions of the stents 210 and 220 installed in the main blood vessel 10 and the branched blood vessel 11 are adjusted by using the catheter according to the present invention, the two guide wires 30 are inserted into the branched-portion ends 21 of the respective guide portions and are then withdrawn through the fixed-portion ends 23.

Thereafter, the catheter is inserted into the main blood vessel 10 and is fixed in a position from which the branched blood vessel 11 branches out, and the guide wires 30 are adjusted such that the branched portion of the first guide portion is pushed so as to be inserted into the main blood vessel 10 and the branched portion of the second guide portion is pushed so as to be inserted into the branched blood vessel 11.

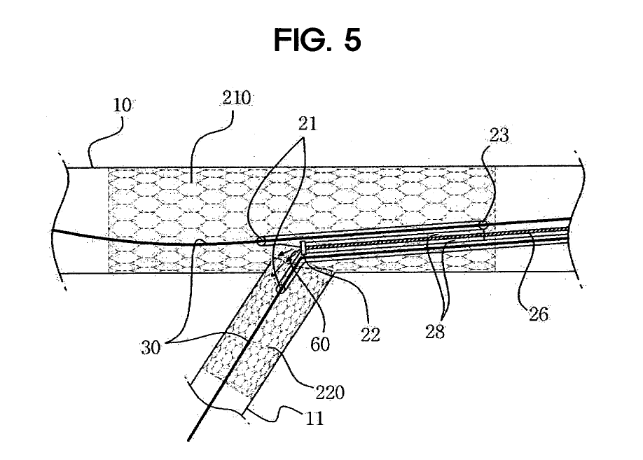

The operator then pressurizes the protrusion by using the main-body front end portion 60 of the catheter, thereby removing the protrusion (see FIG. 5). At this time, the operator may pressurize an accurate carina portion through the radio marker, which is the marking unit 22 positioned in the front end portion 60.

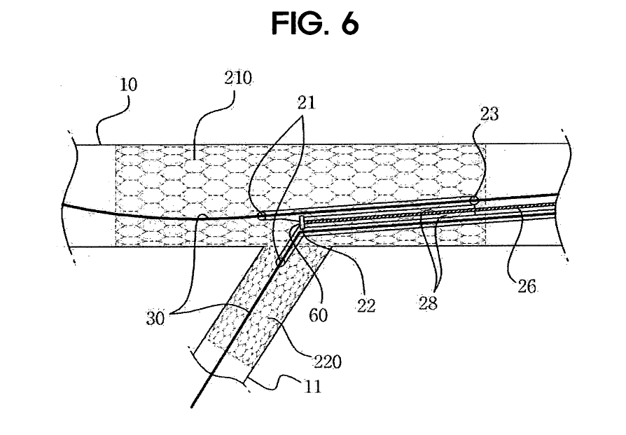

The pressurization may be done several times, and a protrusion 100 is gradually bent toward the vessel wall due to the pressurization (see FIG. 6). Thus, the catheter according to the present invention can prevent instent-restenosis (ISR) caused by the delay of blood flow due to the protrusion 100, and can improve the blood flow by removing the protrusion 100.

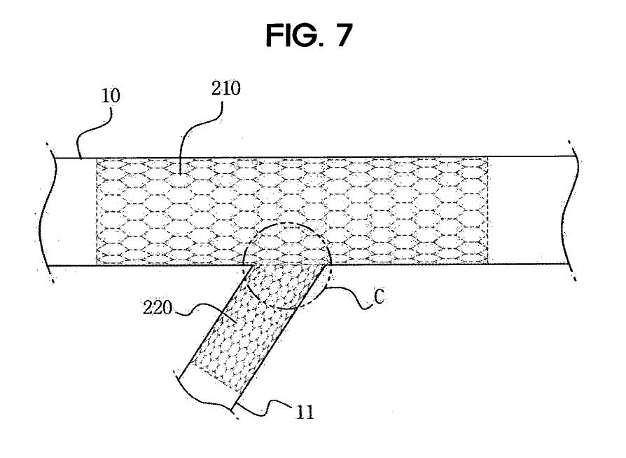

FIG. 7 shows a stent placed in the branched blood vessel, which has an improved structure from which the protrusion is removed by pressurizing the carina portion by means of the carina modification catheter according to the present invention.

Through the foregoing operation, the protrusion formed in installing the stent 220 in the branched blood vessel 11 can be removed (see a portion C of FIG. 7).

1. A carina modification catheter comprising:
a main body; and
a first guide portion and a second guide portion which are fixed to the main body, respectively,
wherein each of the first guide portion and the second guide portion comprises a fixed portion fixed to the main body and a branched portion extending from the fixed portion and bent at a front end portion of the main body, the front end portion being capable of pressurizing a protrusion portion of a stent inserted into a branched blood vessel.

2. The carina modification catheter of claim 1, wherein the fixed portion of each of the first guide portion and the second guide portion is bonded to an outer circumferential surface of the main body.

3. The carina modification catheter of claim 1, wherein the fixed portions of the first guide portion and the second guide portion have lengths which are different from each other.

4. The carina modification catheter of claim 3, wherein a difference between the length of the fixed portion of the first guide portion and the length of the fixed portion of the second guide portion is in a range from 7 cm to 12 cm.

5. The carina modification catheter of claim 1, wherein a diameter of each of the first guide portion and the second guide portion is in a range from 0.014 inches to 0.015 inches.

6. The carina modification catheter of claim 1, wherein the fixed portion of each of the first guide portion and the second guide portion is a tube formed of polyamide, and has a stainless mesh inserted thereinto.

7. The carina modification catheter of claim 1, wherein the branched portion of each of the first guide portion and the second guide portion is a tube formed of polyamide.

8. The carina modification catheter of claim 1, wherein the branched portion of the first guide portion and the branched portion of the second guide portion are inclined at an acute angle with respect to the front end portion of the main body, respectively.

9. The carina modification catheter of claim 8, wherein the branched portion of each of the first guide portion and the second guide portion has a length of 2 mm to 5 mm.

10. The carina modification catheter of claim 1, wherein the main body comprises:
a housing made of a soft material; and
a support member disposed within the housing along a longitudinal direction.

11. The carina modification catheter of claim 10, further comprising a first blocking plate and a second blocking plate which are disposed between the support member and the housing of the main body, the first and the second blocking plates corresponding to the first guide portion and the second guide portion, respectively.

12. The carina modification catheter of claim 10, further comprising a marking unit fixed at a front end portion of the support member.

13. The carina modification catheter of claim 12, wherein the marking unit is formed of a metal or resin which does not pass radiation therethrough.

14. The carina modification of catheter of claim 13, wherein the metal is lead or gold.
